(19) United States
(12) Patent Application Publication
(10) Pub. No.: US 2026/0137542 A1
(43) Pub. Date: May 21, 2026

(54) ORTHOPEDIC BRACE WITH CUSTOM-FITTED FRAMEWORK OF LIGHT-CURABLE MATERIAL

(71) Applicant: Arctic Bracing LLC, McKinney, TX (US)

(72) Inventors: Jeff King, Melissa, TX (US); Brian Snow, Fairview, TX (US)

(21) Appl. No.: 19/396,702

(22) Filed: Nov. 21, 2025

Related U.S. Application Data
(60) Provisional application No. 63/723,403, filed on Nov. 21, 2024.

(51) Int. Cl. A61F 5/01; A61L 31/02; A61L 31/04; A61L 31/06

(52) U.S. Cl. CPC A61F 5/0125; A61L 31/028; A61L 31/048; A61L 31/06; A61F 2005/0167; A61F 2005/0174

(57) ABSTRACT

A customizable component of an orthopedic brace is provided that includes a panel with a flexible substrate and a chamber attached to a surface of the flexible substrate, where the chamber includes an at least semi-transparent shell and is shaped to align with at least one edge of the substrate, where the chamber is to accept and contain a light-curable material.

### RELATED APPLICATIONS

This Application claims benefit to U.S. Provisional Patent Application Ser. No. 63/723,403, filed Nov. 21, 2024, the disclosure of which is incorporated by reference herein in its entirety.

### BACKGROUND

The present disclosure relates in general to the field of orthopedic devices, and more specifically, to customizable orthopedic brace devices.

Orthoses, or orthopedic devices, serve as medical aids for stabilizing, relieving stress, immobilization and, in particular, for guiding or correcting a patient's limbs and joints, including the corresponding muscle tissue, ligaments, and bone structures. Generally, mechanical stabilization and guiding or correction is achieved in particular by mechanically rigid stabilizing elements in the orthopedic devices, which are brought into firm mechanical contact with the body such that supporting forces can be absorbed or correction forces can be exerted. Mechanical joint rails and bridges are often employed, in connection with rigid frames or other structure to provide such protection, correction, and guidance. A range of orthopedic devices have been developed for various parts of the human body (as well as for veterinary uses), including braces for knees, hips, spine, elbow, wrists, ankles, etc.

### BRIEF DESCRIPTION OF THE DRAWINGS

FIG. 1 illustrates an example orthopedic brace.

FIGS. 2A-2B illustrate views of an example brace panel hinge assembly.

FIG. 3 is a side view of an example brace panel hinge assembly.

FIG. 4 is a perspective view of an example brace panel.

FIG. 5 illustrates example fitting of a customized brace device utilizing a light-curable resin in accordance with at least one embodiment.

FIG. 6 is an exploded view of an example orthopedic brace.

FIG. 7 illustrates assembly aspects of an example orthopedic brace.

FIG. 8 is a photographic image illustrating an example orthopedic brace worn by a user.

FIG. 9 illustrate example hinge elements to couple to brace panels in an example orthopedic brace.

FIG. 10 illustrate example support straps for use in an example orthopedic brace.

Like reference numbers and designations in the various drawings indicate like elements.

### DETAILED DESCRIPTION

An improved orthopedic brace is provided in the present disclosure utilizing a panel, which includes one or more hollow, low profile chambers (e.g., formed from respective chamber molds or shells), which are filled with a curable resin capable of being cured using ultraviolet light (UV), to custom fit the panel to the contours of a patient's particular anatomy. The panels may be fastened to padding, soft goods, and/or hinge elements of the orthopedic brace. One or more panels with chambers filled with UV-curable resin may be incorporated into the orthopedic brace to enable custom-fitting of the brace to each patient. This allows for the provision of orthopedic braces (e.g., knee braces, elbow braces, scoliosis braces, ankle braces, hip braces, back braces, wrist braces, etc.) that have a more comfortable fit customized to an individual patient's size, shape, and degree of deformity secondary to the arthritic condition. By applying and molding the custom framework to each patient, the patient's native deformity becomes the brace's shape, for a more natural and comfortable fit. This also sets the brace at the starting anatomy of the patient's native deformity, thus allowing more reproducible degrees of unloading, among other example benefits.

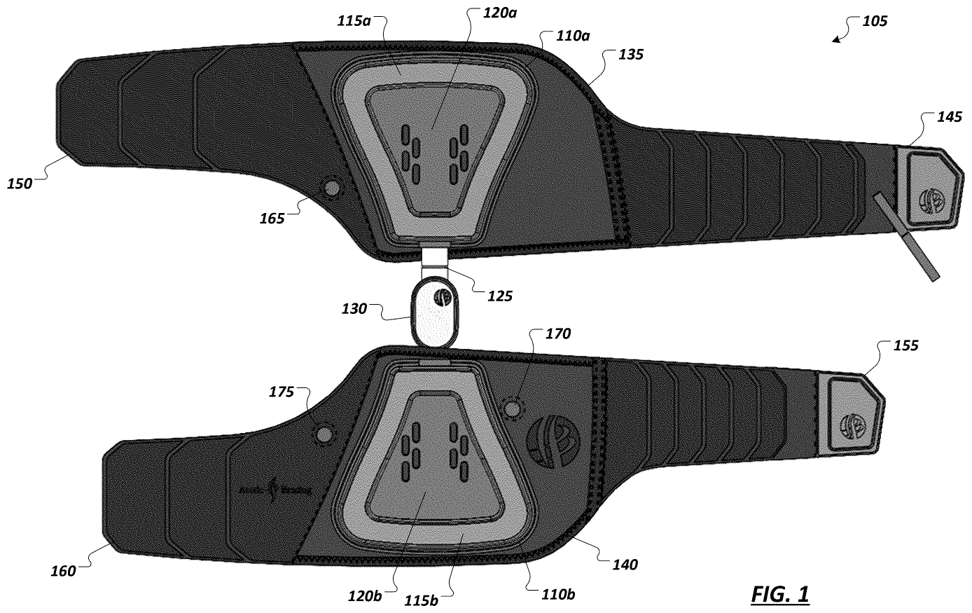

Turning to FIG. 1, an example orthopedic brace 105 is shown implementing a knee brace to support, for instance, the medial compartment, lateral compartment, or other aspects of a human (or animal) knee joint. In this example, two panels 110a, 110b are utilized in the brace to form fulcrum points for supporting a joint with the brace. In other implementations, a single panel or more than two panels may be employed based on the anatomy being supported and the purpose of the brace, among other examples. Panels (e.g., 110a, 110b) are to be coupled to bindings, straps, soft goods, or other material (e.g., 145, 150, 155, 160, etc.) to facilitate the binding or attachment of the brace 105 to its user. In some implementations, to provide a rigid, yet lightweight, customizable, and low profile brace, panels (e.g., 110a, 110b) may be provided with a solid yet flexible substrate (e.g., 120a, 120b) with a chamber mold implementing a bladder, hollow, channel, or other chamber (e.g., 115a, 115b) that is to be attached to or integrate with the surface of the panel. The chamber is to have dimensions that occupy less than all of the surface area of the panel (e.g., 110a, b) (e.g., occupy and cover less than 25%, less 30%, less than 50%, etc. of the overall surface area of the panel). In some implementations, the chamber may be implemented as a low profile chamber element (e.g., 115a, b) with dimensions to align with or correspond to the outside perimeter geometry of the panel (e.g., 110a, b). For instance, in the example of FIG. 1, the panel (e.g., 110a, b) has a roughly trapezoidal shape and perimeter, and the chamber 115a, b is dimensioned and has a shape that corresponds to this outer perimeter of the panel 110a, b. Further, the shape and dimensions of the chamber 115a, b form an opening within this perimeter to expose a portion of the panel's surface (e.g., of substrate 120a, b) not covered by the chamber. As such, a portion of the plate substrate 120a, b (e.g., within the inside perimeter of the chamber mold) is not covered by or connected to the chamber 115a, b.

While the example panels and chambers shown in the example of FIG. 1 (and other examples shown and described herein), may adopt a specific geometry, it should be appreciated that the plate and chambers discussed herein may adopt a variety of geometries and dimensions, as is appropriate to the type and function of brace that is be implemented or constructed using the panels. As examples, a panel may have a substantially circular or oval geometry, a rectangular geometry, a triangular geometry, or an irregular geometry. Panels used in a single brace may have different geometries. Similarly, panel chambers (that are to hold light-curable material) may be implemented with similarly varied geometries to correspond to the geometry of the panel to which they are to be attached or incorporated. For instance, as in the example of FIG. 1, the chamber may be implemented to sit within and correspond to the entire outer perimeter of the panel, but leave at least some portion or portions of the panel substrate uncovered by the chamber. In some implementations, rather than aligning with and tracking the entire outer perimeter of the panel substrate geometry, the chamber element may correspond to only a portion or segment of the overall outer perimeter of the panel (e.g., and leave one or more other segments of the panel's perimeter without a corresponding chamber element). In some implementations, multiple chamber elements may be provided on the surface of the panel substrate (e.g., rather than a single, unitary chamber for a single panel). Indeed, it should be appreciated that various different panel and chamber designs may be adopted and integrated within an improved orthopedic device without departing from the more generalized principles being described and illustrated herein through the more specific example figures and implementations provided.

Continuing with the illustrative example of FIG. 1, the flexible substrates (e.g., 120a, b) of the panels (e.g., 110a, b) by themselves, may not provide the requisite rigidity, strength, and structure to allow a corresponding orthopedic brace to function properly (e.g., to provide suitable leverage for a joint or other anatomy being supported by the brace). The substrate (e.g., 120a, b) may be suitably flexible, however, to allow it to be wrapped or formed around the specific contours of a patient, while providing a thin (low profile) and lightweight panel substrate for the brace. By itself, the substrate's flexibility may not provide sufficient rigidity for the substrate to function alone as a panel for providing a fulcrum point for a brace. To structurally reinforce the substrate (and form a panel of a brace), one or more chamber mold elements (e.g., 115a, b) may be attached to or integrated with the surface of the substrate (e.g., near to and aligned with one or more (or all) perimeter edges of the substrate (e.g., 120a, b)). The chamber mold may be prefilled with or injected to be filled with a UV-curable resin or other light-curable material configured to be cured and hardened through exposure to a UV light source. Before curing, the panel with the chamber mold may be temporarily wrapped and formed around the anatomy of a patient (e.g., during fitting of the brace and under the direction of a medical professional). With the panel wrapped around the patient, the UV light source may be applied to cure and harden the chamber mold to permanently form the panel (e.g., 110a, b) into a desired shape contoured to the anatomy of a given patient, thereby reinforcing the substrate (e.g., 120a, b) of the panel to also act as a fulcrum point for the brace, among other examples.

Continuing with the example of FIG. 1, an orthopedic brace may include one or more hinge elements (e.g., 130) (e.g., to allow two or more portions (e.g., panels or other patient attachment points) to pivot, rotate, or otherwise move in relation to each other). Hinge elements (e.g., 130) may include examples such as monocentric hinges, polycentric hinges, four-bar linkage hinges, elliptical hinges, range-of-motion hinges, ball-and-socket hinges, locking hinges, spring-assisted or resistance hinges, among other examples, as appropriate for the corresponding brace being implemented through inclusion of the light-curable panel elements (e.g., 110a, 110b). On or more arms (e.g., 125) of the hinge 130 may be fastened to one or more of the panel substrates (e.g., 120a, b) to functionally attach the panel(s) (e.g., 110a, 110b) to the hinge 130. The panel substrates may also include or be fastened to bindings (e.g., 145, 150, 155, 160) to enable the panels to be removably fastened to the patient and construct a functioning orthopedic brace (e.g., a knee brace in the particular illustrative example shown in FIG. 1). Some braces may include additional elements to assist in fitting the brace to its user, such as bindings and supplemental support straps (e.g., using button fasteners (e.g., 165, 170, 175), among other example features.

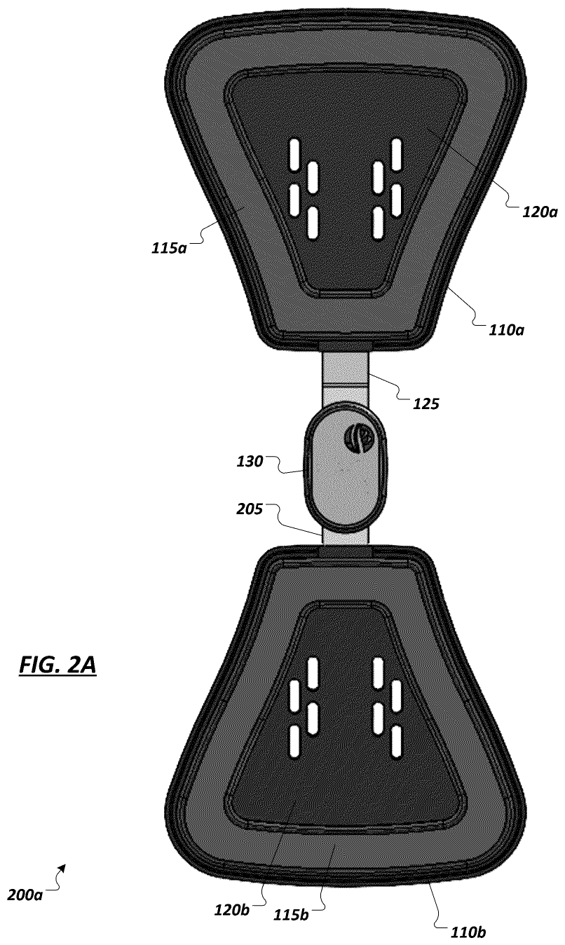

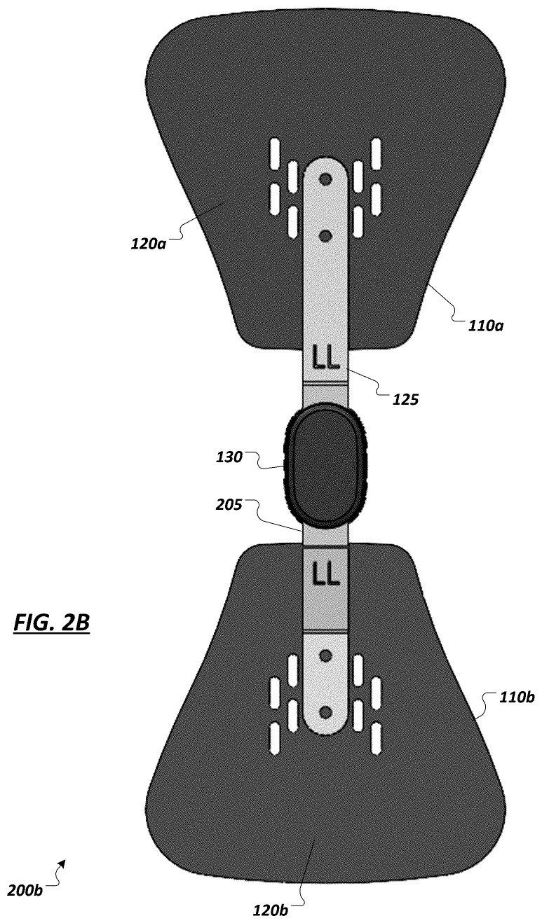

Turning to FIGS. 2A-2B, a front view 200a (in FIG. 2A) and back view 200b (in FIG. 2B) are shown of two example panels 110, 110b connected to a hinge element 130 of an example orthopedic brace (e.g., a knee brace, although other implementations may adopt these same principles to implement other brace types, such as elbow braces, hip braces, ankle braces, foot braces, shoulder braces, etc.). As shown in the example of FIGS. 2A-2B, hinge elements (e.g., 130) and other rigid components of an orthopedic brace may be connected to one or more brace panels (e.g., 110a, b) reinforced through chamber elements (e.g., 115a, b) containing a light-curable resin. In the example of FIGS. 2A-2B, arms (e.g., 125, 205) of an example hinge element 130 are fixedly or permanently secured to the substrates (e.g., 120a, 120b) of the customizable pad plates formed from a UV-curable resin material. For instance, a hinge 130 may include two hinge arms 125, 205 (e.g., constructed from aircraft aluminum, carbon fiber, titanium, or another material), which are each connected to the substrate of a respective one of the panels 110a, b (e.g., using a weld, pseudo rivets, permanent adhesive, fasteners, or other mechanism), such as at a rear side of the panel (e.g., as shown in the illustration of FIG. 2B) or at a front side of the panel (e.g., by sliding the arm underneath the chamber mold and fixing the arm to the substrate surface). In this example, the hinge enables movement (e.g., flexion/extension, abduction, adduction, rotation, circumduction, etc.) and, in some cases, enforces limitations on joint movement (e.g., limiting the range of joint movement). For instance, a Varus/valgus Allen telescoping mechanism may be proved to enable enhanced degrees of correction to the patient's native neutral alignment. For instance, a telescoping Varus/valgus unloading dial may be provided with the hinge 130, which may be rotated to adjust the position of an unloader plate (through telescoping transitional bolts), which may be provided with a pad to adjustably contact the joint (e.g., knee) of the patient. In one example, the hinge is implemented as a two-panel hinge with flexion/extension stop insert elements to enable the range of motion to be controllably limited in correspondence with the determined treatment of the patient, among other example hinges and components which may be incorporated into braces which utilize the brace panels described herein.

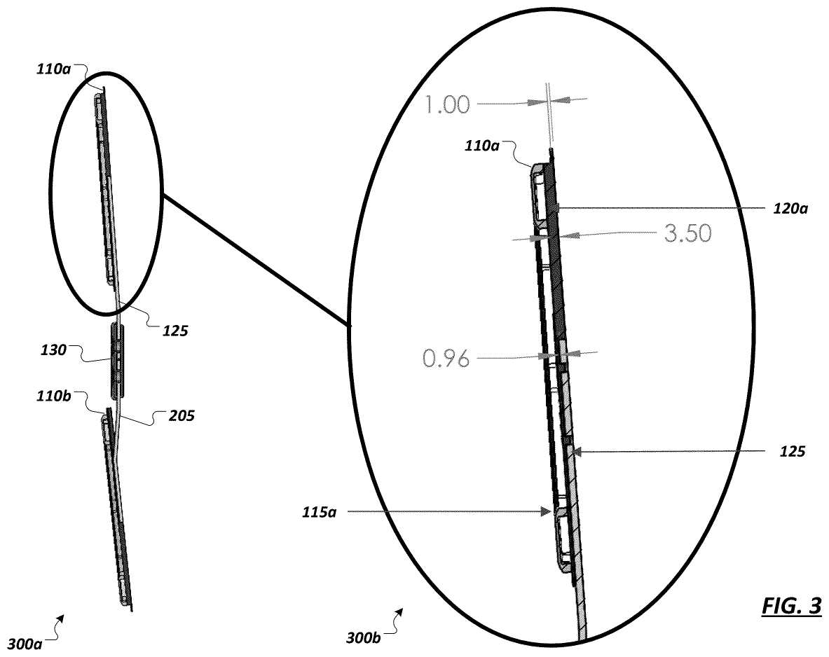

Brace panels constructed from a flexible substrate and reinforced using chamber molds filled with light-curable resin may enable the construction of custom-fitted braces, which are also lightweight and low-profile. For instance, turning to FIG. 3, a side profile view 300a of the components illustrated in FIGS. 2A-2B is shown. More particularly, a side profile view is shown of a portion of an example knee brace including two brace panels 110a, 110b coupled to hinge arms 125, 205 of an example hinge element 130 (e.g., a polycentric hinge). Close-up view 300b shows example depth dimensions of one of the low-profile panels 110a. The angles and dimensions of hinge arms 125, 205 may be selected to implement various knee braces using the same panels 110a, 110b, such as a knee brace for the right or left leg, knee braces for the medial or lateral compartment, etc.

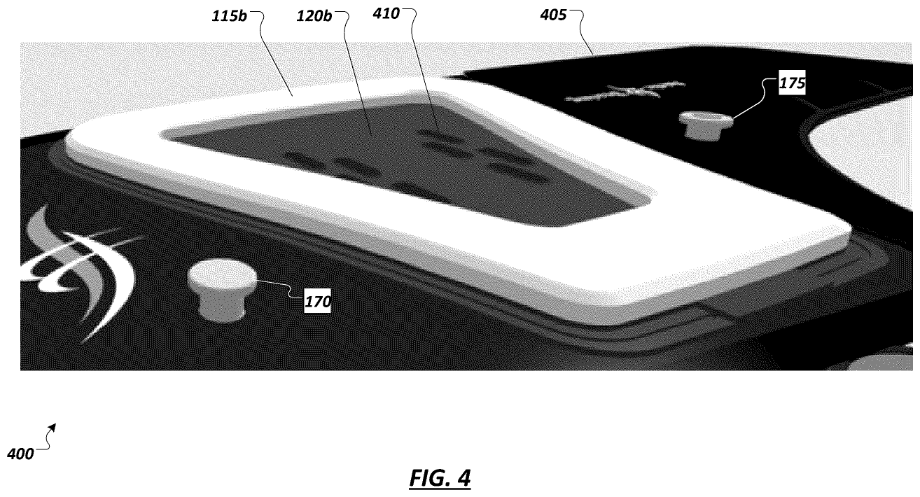

Turning to FIG. 4, a perspective view of an example panel 110b is shown, as attached to a lower leg strap 405 of an example brace. In this example, the panel includes a flexible, solid plastic substrate (e.g., 120b), with a chamber 115b connected to the surface of the substrate. The chamber, in this example has a geometry corresponding to the perimeter geometry of the substrate 120b. The chamber 115b is to be filled with a light-curable resin and permit light to pass through the surface of the chamber shell (when the mold/shell and brace are ready for curing). The shell of the chamber 115b containing the light-curable resin may be formed from a transparent or semi-transparent flexible plastic or other material. The material of the chamber mold 115 is flexible, allowing the chamber to conform to the shape of the flexible substrate to which it is attached, while the panel is formed to the anatomy of a patient prior curing of the UV-curable resin inside the chamber mold 115 and the setting of the brace panels to a particular patient. While a chamber mold could be provided that corresponds to and covers nearly the entire surface of the panel substrate 120b, some implementations of the chamber 115 are shaped and dimensioned to be placed at and conform to strategic portions of the edges or perimeter of the panel substrate 120b. This approach allow sufficient reinforcement of the panel substrate to be achieved, while reducing the overall amount of light-curable resin material used to fill the chamber 115b, allowing the panel to be more breathable (e.g., through openings or vents (e.g., 410) in the substrate 120b, positioned so as not to be covered by the chamber mold 115b), and resulting in lighter weight, thereby reducing the cost and improving the wearability of an orthopedic brace incorporating the panel, among other example advantages.

As discussed above, a UV-curable resin material may be utilized to construct custom-fit panels contoured to the anatomy of specific patients. Such materials may be selected from any composite material that is in a liquid or gel form until polymerized and cured by the energy radiated from ultraviolet irradiation devices. Light-curable resin materials, as discussed herein, may include oligomers, monomers, photo-polymerization initiators, coinitiators (e.g. spectral sensitizer, reducing agents, etc.), and various additives such as stabilizers, antioxidants, plasticizers, and pigments. Light-curable resin materials include acrylate radical polymerization materials (e.g., polyester and epoxy resins, aliphatic and aromatic urethanes, silicones and polyethers) and epoxy cationic polymerization materials, among other examples. The material may be injected or infused within a flexible mold or shell of the chamber, to contain the liquid or gel material and enable forming of the material around the body part of a patient prior to curing. This mold or shell may be adhered to or otherwise attached to a substrate panel to form the chamber. The materials may be a low viscosity epoxy material that includes epoxy resins, acrylate fillers and activators, polyurethane or any combination thereof. In the case of light-curable materials, a light curable composite epoxy resin can be used. Through light curing techniques, the material may be cured to cause the mold or shell containing the UV-curable resin (and any substrate to which the chamber mold is attached) to permanently adopt a particular shape (e.g., corresponding to a body part around which the flexible substrate and chamber mold (containing the material) is wrapped) by exposing the flexible (and at least partially transparent) chamber mold and light-curable material to light. In some implementations, the light-curable material may include epoxy mixed with filler material such as nanofibers to increase strength of cured material. In one implementation, the light-curable material can be a monomer material which is selected to give adequate strengthening for immobilization. Material will be provided as two premix forms which will be mixed prior to being injected or infused into the flexible chamber or bladder. In some examples, polymerization of the material may start within 5-10 minutes of exposure to light and can give basic hardening strength within 15 minutes.

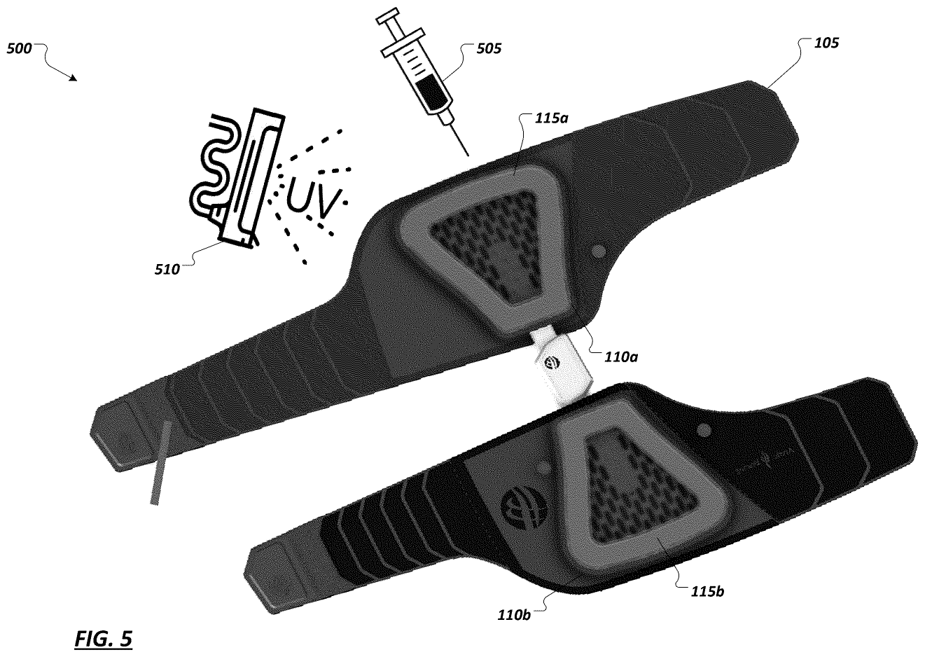

Turning to FIG. 5, a diagram 500 shows the example assembly of a customized brace device 105 utilizing a light-curable resin. A flexible chamber mold (e.g., 115a, 115b) may be provided on a brace panel (e.g., 110a, 110b), each chamber mold forming a flexible, hollow channel or cavity to be filled with and contain a light-curable material and dimensioned to correspond to at least a portion of the outer perimeter of the substrate of a corresponding panel (e.g., 110a, 110b). The chamber molds (e.g., 115a) are adapted to be filled with and contain a light-curable material in its liquid or gel form. For instance, in the example illustrated in FIG. 5, a chamber mold 115a, 115b is shown implementing a low-profile channel corresponding to and aligning with the edge or perimeter of the panel substrate (and omitted from at least a portion of the surface of the panel substrate (e.g., a central portion of the panel fully or substantially circumscribed by the chamber mold element(s))). This portion of the surface of the panel substrate may include openings or venting to improve airflow to the user's skin covered by or in contact with the brace.

The size and shape of panels including a chamber mold filled with light-curable material may vary based on the type and purpose of the corresponding orthopedic brace. While the shape shown in the examples herein may correspond to panels for placement on the legs of a patient in a knee brace, different shaped panels (with chamber molds shaped to conform to the outer perimeter of these panels) may be adopted for other brace types (e.g., scoliosis braces, ankle braces, hip braces, etc.). Further, different sizes and shapes of chamber molds may be provided to accommodate patients of varying ages/sizes and different types of orthopedic braces. The chamber mold may be constructed as a flexible mold, shell, or bladder constructed of material that is at least partially transparent so as to allow light to penetrate the surface of the chamber mold 115a and cure the light-curable material contained within the channels, bladders, etc. of the chamber mold. In some implementations, the flexible chamber mold may be composed of an at least semi-transparent, flexible, elastomeric materials, such as a silicon, silicon rubber, latex rubber, synthetic rubber, or other material or combination thereof. The flexible nature of the material allows the chamber mold to be wrapped, stretched, formed around a patient's limb (or other body part), while allowing UV energy to pass through the chamber mold and cure the light-curable material contained within the chamber.

Continuing with the example of FIG. 5, in some implementations, a chamber mold (e.g., 115a, 115b) may be prefilled with light-curable material. In other instances, the chamber mold is empty and is to be filled (e.g., nearer to the time of fitting) with the light curable materials (e.g., to limit the opportunities for the material to be accidentally exposed to light prior to fitting). For instance, the light curable material may be injected 505 into the chamber mold 115a and the injection hole sealed (e.g., using laser sealing, an adhesive, or sealant) following filling of the chamber mold 115a. The filling of the chamber mold within the light-curable material may be performed in a space shielded from UV light. Prior to fitting and curing (e.g., during transport and storage), the chamber mold may be covered by a removable film, coating, or other covering, which inhibits light from passing into the chamber mold before the chamber mold is ready to be cured (e.g., during fitting of a corresponding orthopedic brace). Such film may be removed when a corresponding brace is attached to a patient and the chamber molds and panels of the brace are to be exposed to light (e.g., from a UV light source 510) to cure the light-curable resin within the chambers and thereby harden the panels to the contours of the brace's wearer. In some instances, the chamber mold (e.g., 115a) for a panel (e.g., 110a) may be a single continuous (fluidically connected) chamber mold. In other instances, the chamber mold may be composed of or form two or more separate cavities, which are to be separately filled, among other example implementations.

In some implementations, infusion of light-curable material (in its liquid or gel form) within the internal channels of a flexible brace plate chamber mold (e.g., 115a, 115b) may be performed utilizing a syringe (e.g., 505) or other pump device (e.g., electrically or mechanically powered) to pump the light-curable material into the chamber mold 115a, b. UV light (e.g., 510) or other suitable light energy may then be applied to the chamber mold to cure the material within the chamber and set the shape of the chamber mold (and internal material) to conform to the body of a specific patient (while the underlying substrate is formed around a limb, hip, back, neck, or other body part of the patient). In some implementations, while the chamber mold may be transparent, a removable covering or layer may be provided on the exterior of the chamber mold to protect light-curable material within from being prematurely exposed to light and cured. In such examples, the covering may be removed to enable light 510 exposure to the chamber mold 115a, b and curing of the internal light-curable material. For instance, the chamber mold infused with the material may be wrapped around a leg (or other body part) of a patient under the supervision of a medical professional. When the chamber mold is positioned as desired, light may be introduced to cause the material to cure and harden to permanently form the chamber mold into a resulting panel of a brace.

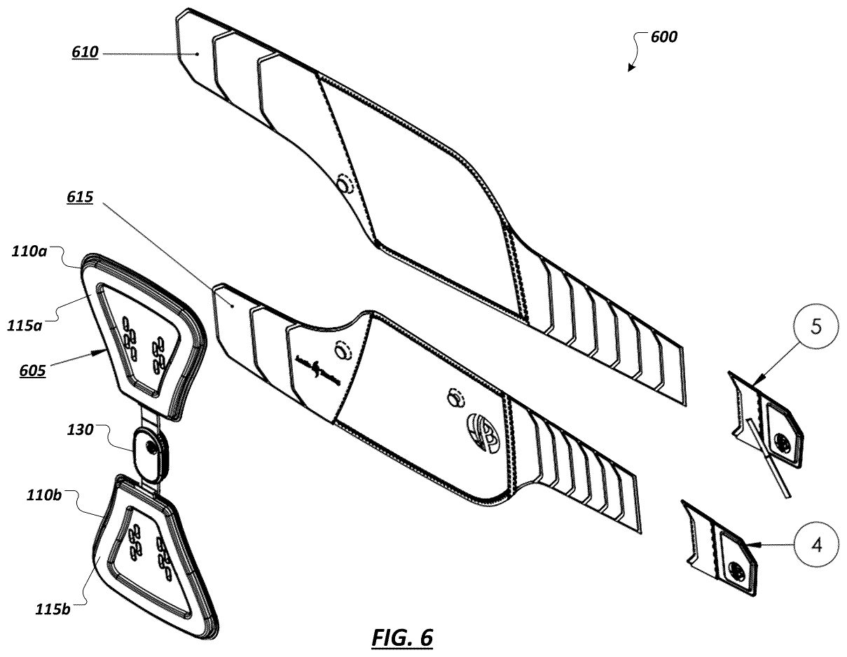

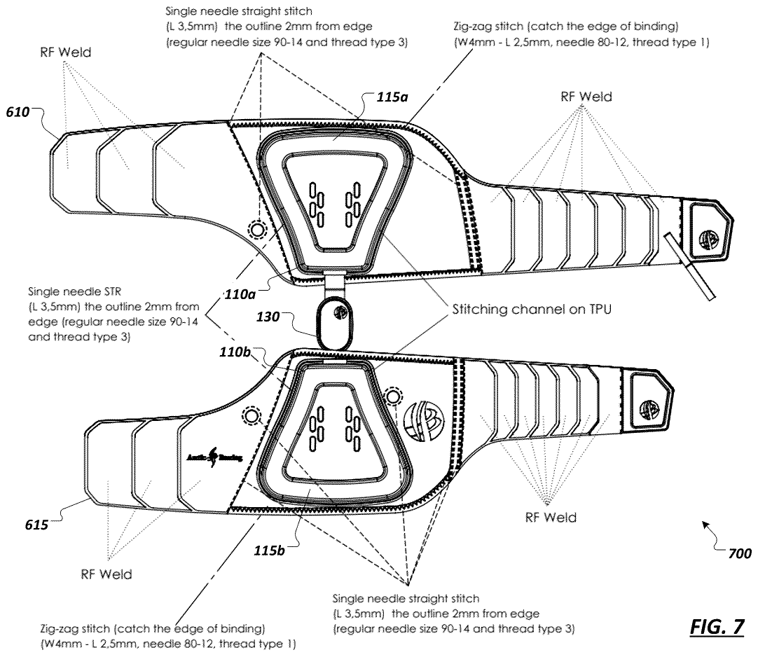

FIG. 6 is an exploded view 600 of an example brace (e.g., similar to the example brace shown in FIG. 1), showing a panel-hinge assembly 605 including brace panels 110a, b with corresponding chamber molds 115a, b including (or to be filled with) light curable material. The two panels 110a, 110b are connected to respective arms of a hinge 130. The assembly 605 may be attached to bindings 610, 615 configured to secure the panels of the brace to respective parts of the patient's anatomy. For instance, in the example of a knee brace, a first binding may be used to secure a first brace panel to the leg above the knee joint and a second binding may be used to secure the second brace panel to the leg below the knee joint of the patient, among other examples. FIG. 7 shows a front view 700 of the example assembly of the example brace 105. In some implementations, the substrate of the panels 110, 110b are sewn (e.g., at an outer edge of the panel substrates) to respective bindings 610, 615 to securely fasten the panel hinge assembly 605 to the bindings 610, 615 and form the brace 105. Fasteners (e.g., 165, 170, 175), such as hook-and-loop fasteners, button fasteners, buckled fasteners, and other fasteners may be incorporated within the bindings to enable securing of the bindings to the anatomy of the user. In the example of FIG. 7, button fasteners (e.g., 165, 170, 175) are shown. Fasteners and other elements may also be attached to the bindings 610, 615 (e.g., through sewing, adhering, or otherwise fastening the fastener to the bindings), among other example components of the brace 105.

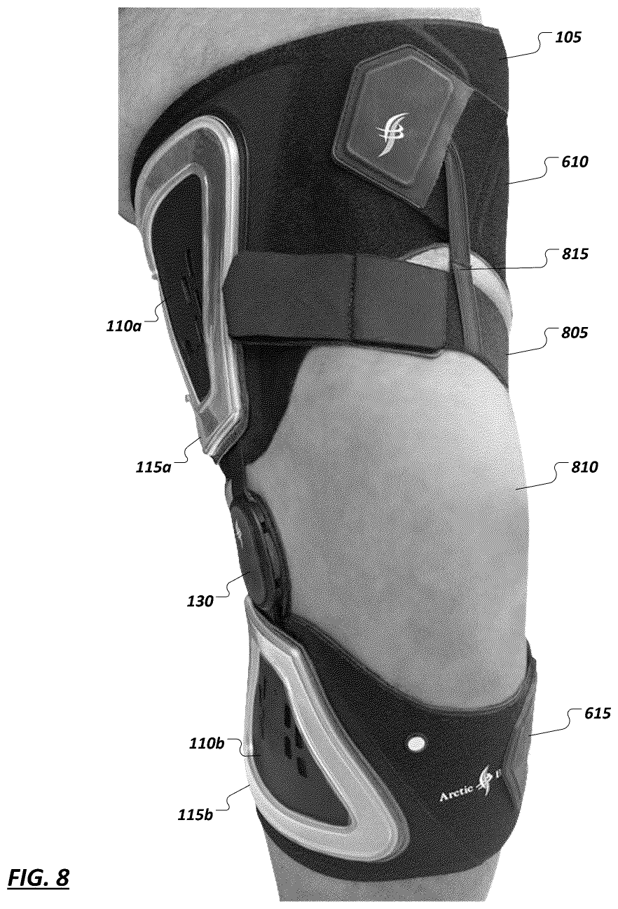

FIG. 8 is a photographic image of the example brace 105 (e.g., introduced in the example of FIG. 1) worn by a user. In this example, an additional support strap 805 is provided in addition to bindings 610, 615, which may be utilized to provide additional stability for the knee joint. The support strap 805 may utilize button fasteners provided on the bindings 610, 615 of the brace 105 to couple to the brace, and wrap from a button fastener located on binding 615 near the calf on the medial side of the patient's leg around the back of the patient's leg and over the leg above the knee 810 to connect to another button fastener provided on the bracing 610 (near the medial side of the knee), among other examples.

As shown in FIG. 8, the bindings 610, 615 may secure the brace 105 to the body of the patient and align the brace appropriately to a joint or other anatomy of the patient, in accordance with the brace's design and purpose (e.g., to align hinge 130 to the medial side of the patient's knee 810). Fitting of the brace to a patient may involve a medical professional supervising the placement of the brace on the patient to ensure the elements of the brace, including panels 110a, 110b, are properly aligned to the patient's anatomy. When properly positioned, the brace may be secured to the patient using the bindings to maintain the alignment of the panels to the patient's body (and properly form the still flexible panels around the patient) prior to curing. With the brace properly positioned and secured, the chamber molds may be exposed to a light source (e.g., by the medical professional supervising the fitting) to permanently cure and set the chamber molds 115a, 115b into a particular contour corresponding to the patient's anatomy. The curing of the chamber molds 115a, 115b also causes the shape of the underlying flexible panel substrate to be fixed into place (along these same contours). Following curing, other adjustments may also be made (e.g., by the medical professional) to custom-fit the brace 105 to the patient, such as adjusting the length and dimensions of the bindings 610, 615 of the brace, adjusting elements of the hinge 130, adjusting the length of one or more support straps (e.g., 805), among other example adjustments. The custom-fit brace 105 is now available for use and reuse by the patient with attributes customized to the patient to maximize their comfort and the overall effectiveness of the brace.

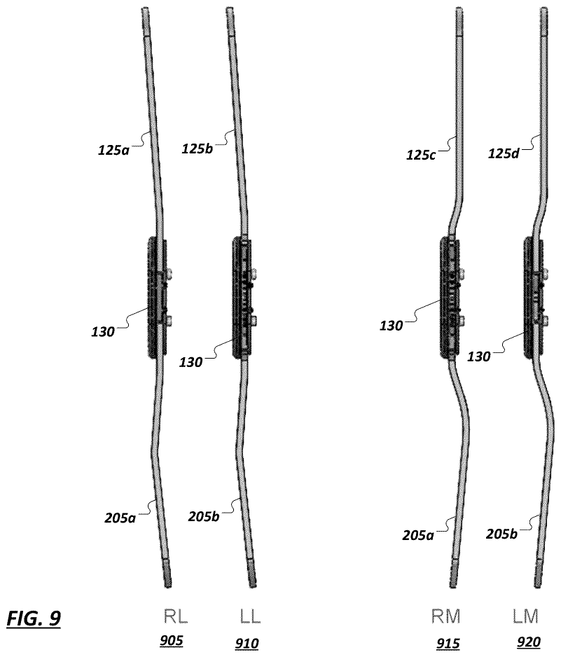

FIG. 9 illustrates side views of example hinge assemblies 905, 910, 915, 920 with different respective geometries, which may be utilized in various knee brace implementations. Brace panels, such as shown and discussed above, may be attached to the respective arms 125a-d, 205a-d of the hinge assemblies 905, 910, 915, 920. Indeed, the same panels may be used with each of the hinge assemblies 905, 910, 915, 920 to alternatively implement various brace designs. For instance, the geometry of hinge assembly 905 may correspond to a lateral knee brace for a right leg, while hinge assembly 910 is used to implement a lateral knee brace for a left leg. Similarly, hinge assembly 915 may be configured for use in implementing a medial knee brace for a right leg, while hinge assembly 920 is used to implement medial knee brace for a left leg, among other examples.

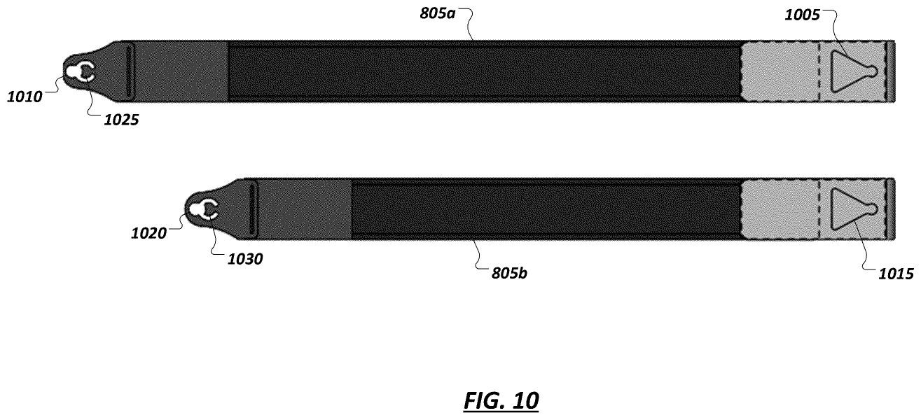

FIG. 10 illustrates example support straps 805a, 805b, which may be used in connection with an example brace device. Support straps may be provided to provide at least some of the supportive structure for a brace. In some implementations, button fasteners may be provided on components of the brace (e.g., binding of the brace, brace panels, etc.) and these button fasteners may be used as attachment points for support straps (e.g., 805a, b) of an orthopedic brace. For instance, ends of a support strap may include keyhole openings 1005, 1010, 1015, 1020 to attach to raised button fasteners of the brace. Some of the keyhole openings (e.g., 1005, 1015) may be dynamic in that they allow for easy and convenient attachment and detachment to one of the button fasteners (e.g., in that the keyhole is simply slipped over the button fastener and secured when tension is applied at the support strap (e.g., 805a, b) and may be freely removed by applying opposite tension at the end of the strap and lifting the keyhole opening away from the button fastener. Other keyhole openings (e.g., 1010, 1020) may provide fixed or static couplings to button fasteners, by providing a lock tab (e.g., 1025, 1030) at the keyhole opening, which is to lock the keyhole opening to a corresponding button fastener (which may be disengaged by depressing the lock tab to remove the keyhole from the button fastener), among other examples.

It should be appreciated that the example knee braces illustrated in the examples above is provided for example purposes only and that brace panels with chamber molds filled with light-curable material may be utilized in a variety of other, different orthopedic devices, such as knee braces of different designs, shoulder braces, hip braces, elbow braces, wrist braces, ankle braces, back braces, etc. Such light-cured braces may likewise utilize various straps and connectors to connect the customized pad plates to the limb (e.g., arm or leg) of the user, among other example features.

Orthopedic braces utilizing customizable, light-curable panels may include points of attachment (e.g., at the panel substrate, such as shown in the example of FIG. 2B) to couple the panels to hinge components, rotational components, or other structural and functional components of the brace device. In some implementations, a hinge (e.g., via its hinge frame members) or other brace component may be removably coupled to a panel with a light-curable chamber mold. This may be advantageous in allowing a faulty component to be replaced and the customized, light-cured pad plates to be reused with the replacement component (e.g., a replacement hinge component) by detaching the faulty component from the panels and attaching the replacement component to the same custom-fitted panels. Similarly, as a user's body grows, atrophies, or otherwise changes in shape or size over time, the cured fitted panels may no longer fit as snugly or comfortably as when the panels were originally (previously) formed around the limb of the user and cured. Accordingly, a hinge member or other component of the brace, which was coupled to the outgrown panels may be reused with new panels, which may be refit to the user's current body and then attached to the original brace component (e.g., a rotational hinge member), among other example uses.

It should be appreciated that while some of the examples shown and illustrated refer specifically to a knee brace, the principles discussed above may be applied equally to other brace types which include one or more panels coupled to one or more hinges or controlled movement mechanisms. Examples of such braces may include, for instance, elbow braces, hip braces, shoulder braces, wrist braces, back braces, foot braces, and ankle braces. These concepts and solutions may also be applied to bracing solutions for animals (e.g., in veterinary medicine), among other example implementations of these principles.

Any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. In addition, unless stated otherwise, a set of elements may include one or more elements.

As used herein, the term “substantially” in reference to a given parameter, property, or condition means and includes to a degree that one of ordinary skill in the art would understand that the given parameter, property, or condition is met with a small degree of variance, such as, for example, within acceptable manufacturing tolerances. By way of example, depending on the particular parameter, property, or condition that is substantially met, the parameter, property, or condition may be at least 90% met, at least 95% met, or even at least 99% met.

In the drawings, while some structural or method features may be shown in specific arrangements or orderings, such specific arrangements or orderings may be optional. In some examples, such features may be arranged in a different manner or order than shown in the drawings. Additionally, the inclusion of a structural or method feature in a particular drawing is not meant to imply that such feature is present in all examples, and, in some examples, such feature may not be included or may be combined with other features. Further, the various examples shown in the drawings are illustrative representations and may not be drawn to scale. In some instances, the same or similar reference numerals may be used to designate the same or similar features in different drawings.

Particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

Thus various examples have been described herein. For instance, example 1 is an orthopedic brace including: a panel including a substrate and a mold aligned with at least one edge of the substrate, where the mold is to accept and contain a light-curable material; and a hinge including one or more hinge framework elements coupled to the substrate of the panel.
Example 2 includes the subject matter of example 1, where light-curable material includes an ultraviolet-light-curable resin.
Example 3 includes the subject matter of any one of examples 1-2, where the substrate includes a flexible substrate and the mold is mounted to a surface of the flexible substrate.
Example 4 includes the subject matter of any one of examples 1-3, where the one or more hinge framework elements are removably coupled to the substrate.
Example 5 includes the subject matter of any one of examples 1-4, where the one or more hinge framework elements are coupled to be flush with a surface of the flexible substrate.
Example 6 includes the subject matter of any one of examples 1-5, where the substrate has a geometry with an outer perimeter, the mold is shaped to correspond to the outer perimeter.
Example 7 includes the subject matter of example 6, where the mold covers an area of a front surface of the substrate corresponding to the outer perimeter, and the mold forms a perimeter around an uncovered area of the front surface of the substrate.
Example 8 includes the subject matter of any one of examples 1-7, where the light-curable material is cured within the mold to fix the flexible substrate in an orientation, where the orientation corresponds to an anatomy of a patient.
Example 9 includes the subject matter of any one of examples 1-8, where the mold is formed from a silicon-based material.
Example 10 includes the subject matter of any one of examples 1-9, where the brace includes one of a knee brace, elbow brace, should brace, hip brace, back brace, wrist brace, shoulder brace, or ankle brace.
Example 11 includes the subject matter of any one of examples 1-10, where the light-curable material includes a ultraviolet (UV) light-curable resin.
Example 12 includes the subject matter of example 11, where the UV light-curable resin includes one of an acrylate radical polymerization material or an epoxy cationic polymerization material.
Example 13 is an apparatus including: a panel for use in an orthopedic brace, where the panel includes: a flexible substrate; and a mold attached to a surface of the flexible substrate, where the mold is aligned with at least one edge of the substrate, where the mold is to accept and contain a light-curable material.
Example 14 includes the subject matter of example 13, where the panel includes connection points to couple to one or more components of the orthopedic brace.
Example 15 includes the subject matter of example 14, where the one or more components includes a hinge arm of the orthopedic brace.
Example 16 includes the subject matter of any one of examples 13-15, further including the light-curable material.
Example 17 includes the subject matter of any one of examples 13-16, where the mold is shaped to correspond with an outer perimeter of the flexible substrate.
Example 18 includes the subject matter of any one of examples 13-17, where the flexible substrate is constructed of a thin rigid plate that is conformable to an anatomy of a patient.
Example 19 is a method including: connecting a panel to a hinge element of an orthopedic brace, where the panel includes an elastomeric shell shaped to correspond to one or more edges of the panel, and the elastomeric shell contains a light-curable material; forming the panel to correspond to contours of a body part of a user; and applying ultraviolet light to the elastomeric shell to cure the light-curable material and permanently fix the shape of the panel to correspond to the contours of the body part of the user.
Example 20 includes the subject matter of example 19, where the panel is coupled to a hinge member of the orthopedic brace.
Example 21 includes the subject matter of any one of examples 19-20, further including injecting the light-curable material into the elastomeric shell.
Example 22 includes the subject matter of any one of examples 19-21, where the method includes a method for making the orthopedic brace.
Example 23 includes the subject matter of example 22, where the orthopedic brace includes the orthopedic brace of any one of examples 1-12.

A detailed description has been given with reference to specific exemplary embodiments. It will, however, be evident that various modifications and changes may be made thereto without departing from the broader spirit and scope of the invention as set forth in the appended claims. The specification and drawings are, accordingly, to be regarded in an illustrative sense rather than a restrictive sense. Furthermore, the foregoing use of embodiments and other exemplarily language does not necessarily refer to the same embodiment or the same example, but may refer to different and distinct embodiments, as well as potentially the same embodiment.

1. An orthopedic brace comprising:
a panel comprising a substrate and a chamber attached to the substrate, wherein the chamber is shaped to align with at least one edge of the substrate, wherein the chamber is to accept and contain a light-curable material; and
a hinge comprising one or more hinge framework elements coupled to the substrate of the panel.

2. The orthopedic brace of claim 1, wherein light-curable material comprises an ultraviolet (UV)-light-curable resin.

3. The orthopedic brace of claim 2, wherein the UV-light-curable resin comprises one of an acrylate radical polymerization material or an epoxy cationic polymerization material.

4. The orthopedic brace of claim 1, wherein the substrate comprises a flexible substrate and the chamber is attached to a front side surface of the substrate, and a back side surface of the substrate is to couple to a binding.

5. The orthopedic brace of claim 4, wherein the one or more hinge framework elements are coupled at the back side surface of the substrate to be flush with the back side surface of the substrate.

6. The orthopedic brace of claim 1, wherein the substrate has a geometry with an outer perimeter, the chamber is shaped to correspond to the outer perimeter.

7. The orthopedic brace of claim 6, wherein the chamber covers an area of a front surface of the substrate corresponding to the outer perimeter, and the chamber forms a perimeter around an uncovered area of the front surface of the substrate.

8. The orthopedic brace of claim 7, wherein the uncovered area of the front surface of the substrate comprises one or more ventilation openings.

9. The orthopedic brace of claim 1, wherein the light-curable material is cured within the chamber to fix the substrate and the panel in a three-dimensional orientation corresponding to an anatomy of a patient.

10. The orthopedic brace of claim 1, wherein the chamber comprises a transparent shell formed from a silicon-based material.

11. The orthopedic brace of claim 1, wherein the orthopedic brace comprises one of a knee brace, elbow brace, shoulder brace, hip brace, back brace, wrist brace, or ankle brace.

12. An apparatus comprising:
a panel for incorporation as a component of an orthopedic brace, wherein the panel comprises:
a flexible substrate; and
a chamber attached to a surface of the flexible substrate, wherein the chamber comprises an at least semi-transparent shell and is shaped to align with at least one edge of the flexible substrate, wherein the chamber is to accept and contain a light-curable material.

13. The apparatus of claim 12, wherein the panel comprises connection points to couple to one or more solid hardware components of the orthopedic brace.

14. The apparatus of claim 13, wherein the one or more solid hardware components comprises an arm of a hinge assembly for the orthopedic brace.

15. The apparatus of claim 12, further comprising the light-curable material.

16. The apparatus of claim 12, wherein the chamber is shaped to form a closed interior border corresponding to and within an outer perimeter of the flexible substrate.

17. A method comprising:
connecting a panel to a hinge element of an orthopedic brace, wherein the panel comprises a substrate and an elastomeric shell coupled to a surface of the substrate and shaped to correspond to one or more edges of the substrate;
forming the panel to correspond to contours of a body part of a user; and
applying ultraviolet light to the elastomeric shell to cure a light-curable material within the elastomeric shell and permanently fix contours of the panel to correspond to the contours of the body part of the user.

18. The method of claim 17, further comprising injecting the light-curable material into the elastomeric shell.

19. The method of claim 17, wherein the panel is attached to a brace binding and the brace binding is used to secure the panel to the body part of the user to form the panel to the contours of the body part of the user.

20. The method of claim 19, further comprising constructing an orthopedic brace comprising the panel, the hinge element, and the brace binding.
